(19) United States
(12) Patent Application Publication
(10) Pub. No.: US 2023/0244950 A1
(43) Pub. Date: Aug. 3, 2023

(54) DISTRIBUTIONS OVER LATENT POLICIES FOR HYPOTHESIZING IN NETWORKS

(71) Applicant: BenevolentAI Technology Limited, London (GB)

(72) Inventors: Daniel Lawrence NEIL, Williamsburg, VA (US); Dane Sterling CORNEIL, Brooklyn, NY (US)

(73) Assignee: BenevolentAI Technology Limited, London (GB)

(21) Appl. No.: 18/193,722

(22) Filed: Mar. 31, 2023

Related U.S. Application Data
(63) Continuation of application No. PCT/GB21/52431, filed on Sep. 20, 2021.
(60) Provisional application No. 63/086,903, filed on Oct. 2, 2020.

(51) Int. Cl. G06N 3/092; G06N 3/0475

(52) U.S. Cl. CPC G06N 3/092; G06N 3/0475

(57) ABSTRACT

Embodiments of present disclosure provide a system, apparatus and method(s) for determining one or more target nodes and associated paths from a query of a graph structure. The method receives the query to the graph structure, where the query comprises a data representation of at least one query node. The method identifies one or more target nodes in response to the query based on a policy network, where the policy network is configured to determine the one or more target nodes in accordance with a latent policy distribution associated with the policy network. The method traverses the graph structure by a search in relation to the policy network, where the search is configured to navigate from the query node to the one or more identified target nodes to determine the associated paths. The method outputs a list of the one or more target nodes and the associated paths for the query, where the list are ranked in relation to the latent policy distribution.

### CROSS-REFERENCE TO RELATED APPLICATIONS

This application is a bypass continuation of International Application No. PCT/GB2021/052431, filed on Sep. 20, 2021, which in turn claims the priority benefit, under 35 U.S.C. 119(e), of U.S. Application No. 63/086,903, filed on Oct. 2, 2020. Each of these applications is incorporated herein by reference in its entirety for all purposes.

### FIELD OF INVENTION

The present application relates to a system, apparatus and method(s) for training and applying the trained policy network for traversing a graph structure to determine a ranked list of targets and associated paths.

### BACKGROUND

It is becoming increasingly common to use knowledge graphs to infer targets (i.e. genes) for diseases and biological mechanisms. One approach is to use a multi-hop method, in which an agent navigates along multi-step paths through the knowledge graph, traversing from a “query” entity to an “answer” entity. When the agent is trained to navigate from a query entity representing disease or biological mechanism to “answer” entities representing targets related to that disease or mechanism, it can learn (with sufficient training) the types of multi-step paths that predict novel therapeutic targets. This is typically accomplished with reinforcement learning, in which the agent is rewarded for successfully navigating from the query entity to an “answer” entity.

There are several issues with existing multi-hop methods, one of which is that there are many possible paths in a graph from the query to a target; the agent may become focused on spurious paths (i.e. paths that exist in the graph between the query and a target but don't actually correspond to a useful or generalisable chain of reasoning) early in training. This has been addressed previously by action dropout methods where links in the knowledge graph are masked, which forces the agent to explore multiple paths with the aim of discovering more “good” paths, paths representing generalisable reasoning, without becoming stuck on “bad” paths, spurious paths.

Although action dropout (randomly masking links between entities as the agent explores) forces the agent to explore multiple paths, it does not incentivise the agent to explore multiple targets. Another issue is that the agent is only rewarded for finding known (benchmark) targets related to the disease, when in fact many more targets may be related to the disease but are unknown. This has previously been addressed by using reward shaping, where a pre-trained graph tensor factorisation model is used to determine the reward given to the out-of-benchmark targets. The tensor factorisation model provides an estimate that an out-of-benchmark target is actually related to the disease; this improves the agent's ability to learn outside of the rare instances when it finds a known benchmark target, and avoids over-penalising the agent for reasonable predictions. A problem with this is that the model is biased towards making the same prediction as the pre-trained tensor factorisation model, rather than using data in independent and complementary ways (to make valid predictions that are missed by a tensor factorisation approach). As an engineering issue, multi-hop algorithms also typically need to truncate the agent's possible action space to N neighbours of the current entity due to memory constraints, where the N neighbours are chosen to be those that are the most connected. For example, if an entity is connected to 10,000 neighbours in the graph and N=200, the agent is restricted to only consider the 200 most highly connected neighbours to traverse to. This limits the data that the algorithm can learn from, and biases the agent towards travelling through highly connected entities.

For these above reasons, it is desired to develop a method, system, medium and/or apparatus, collectively coined as DOLPHIN (Distributions over Latent Policies for Hypothesising in Networks), that can address at least the above issues and produce higher quality paths and perform well on data-rich diseases.

The embodiments described below are not limited to implementations which solve any or all of the disadvantages of the known approaches described above.

### SUMMARY

This Summary is provided to introduce a selection of concepts in a simplified form that are further described below in the Detailed Description. This Summary is not intended to identify key features or essential features of the claimed subject matter, nor is it intended to be used to determine the scope of the claimed subject matter; variants and alternative features which facilitate the working of the invention and/or serve to achieve a substantially similar technical effect should be considered as falling into the scope of the invention disclosed herein.

The present disclosure provides a method, system, medium and/or apparatus, describes DOLPHIN (Distributions Over Latent Policies for Hypothesizing in Networks). DOLPHIN adapts to a trained policy network that can be used for navigating amongst node entities on a graph structure via an agent. From an input query, DOLPHIN provides an output that ranks a set of targets (nodes) and associated paths. In response to the input query, the agent of the policy network traverses the graph structure to identify the set of targets based on the trained policy network. The trained policy network or a set of policies determines the agent's movement on the graph structure. Each policy of the set may be optimised by training to increase the likelihood of navigating through the graph structure to a training target or object entity that is fitted to establish a probability distribution over the set of policies.

In a first aspect, the present disclosure provides a computer-implemented method for determining one or more target nodes and associated paths from a query of a graph structure, comprising: receiving the query to the graph structure, wherein the query comprises a data representation of at least one query node; identifying one or more target nodes in response to the query based on a policy network, wherein the policy network is configured to determine the one or more target nodes in accordance with a latent policy distribution associated with the policy network; traversing the graph structure by a search in relation to the policy network, wherein the search is configured to navigate from the query node to the one or more identified target nodes to determine the associated paths; and outputting a list of the one or more target nodes and the associated paths for the query, wherein the list are ranked in relation to the latent policy distribution.

In a second aspect, the present disclosure provides a computer-implemented method for generating a policy network from a graph structure for use in the computer implemented method of any preceding claim, the computer-implemented method comprising: receiving a first policy, wherein the first policy comprises a set of policies with each policy conditioned on a training triple in relation to the graph structure; optimising the first policy to generate a second policy by minimising entropic differences between the set of policies of the first policy; and establishing the policy network based on the generated second policy in relation to a latent policy distribution.

In a third aspect, the present disclosure provides an apparatus for determining a ranked list of targets and associated paths, the apparatus comprising: an input component configured to receive a query to the graph structure, wherein the query comprises a data representation of at least one query node; a processing component configured to identify one or more target nodes in response to the query based on a policy network, wherein the policy network is configured to determine the one or more target nodes in accordance with a latent policy distribution associated with the policy network; a reactive component configured to traverse the graph structure by a search in relation to the policy network, wherein the search is configured to navigate from the query node to the one or more identified target nodes to determine the associated paths; and an output component configured to output a list of the one or more target nodes and the associated paths for the query, wherein the list are ranked in relation to the latent policy distribution.

The methods described herein may be performed by software in machine-readable form on a tangible storage medium e.g. in the form of a computer program comprising computer program code means adapted to perform all the steps of any of the methods described herein when the program is run on a computer and where the computer program may be embodied on a computer-readable medium. Examples of tangible (or non-transitory) storage media include disks, thumb drives, memory cards etc. and do not include propagated signals. The software can be suitable for execution on a parallel processor or a serial processor such that the method steps may be carried out in any suitable order, or simultaneously.

This application acknowledges that firmware and software can be valuable, separately tradable commodities. It is intended to encompass software, which runs on or controls “dumb” or standard hardware, to carry out the desired functions. It is also intended to encompass software which “describes” or defines the configuration of hardware, such as HDL (hardware description language) software, as is used for designing silicon chips, or for configuring universal programmable chips, to carry out desired functions.

The preferred features may be combined as appropriate, as would be apparent to a skilled person, and may be combined with any of the aspects of the invention.

### BRIEF DESCRIPTION OF THE DRAWINGS

Embodiments of the invention will be described, by way of example, with reference to the following drawings, in which:

FIG. 1a is a flow diagram illustrating an example of applying a policy network to determine a list of targets and associated paths according to the invention;

FIG. 1b is a flow diagram illustrating an example of training the policy network according to the invention;

FIG. 2a is a schematic diagram illustrating an example of the policy network trained and applied to determine a list of targets with associated paths according to the invention;

FIG. 2b flow diagram illustrating another example of the policy network trained via an agent and applied to generate a list of targets with associated paths according to the invention;

FIG. 2c is a schematic diagram illustrating another example of the policy network trained and applied to determine a list of targets with associated paths according to the invention;

FIG. 3 is a schematic diagram of a unit example of a subgraph of the knowledge graph that may be used by the process(es) of FIGS. 1a, 1b, 2a, 2b, and 2c;

FIG. 4 is a block diagram of a computing device suitable for implementing embodiments of the invention.

Common reference numerals are used throughout the figures to indicate similar features.

### DETAILED DESCRIPTION

Embodiments of the present invention are described below by way of example only. These examples represent the suitable modes of putting the invention into practise that are currently known to the applicant, although they are not the only ways in which this could be achieved. The description sets forth the functions of the example and the sequence of steps for constructing and operating the example. However, the same or equivalent functions and sequences may be accomplished by different examples.

The inventive method, system, medium and/or apparatus, describes DOLPHIN (Distributions Over Latent Policies for Hypothesizing in Networks) that adapts a policy network (comprising a set of paths) that is trained and used for navigating from disease/biological mechanism entities to a set of targets associated with the input to a graph structure by potentially an agent. The input to the graph structure is the query comprising query entities of subject and relation entities to be predicted—i.e. disease or biological mechanism and relation—and the output is a ranked set of targets and paths predicted to have the relation to the query entities. In response to the input query, the agent of the policy network traverses the graph structure to identify targets based on a set of policies underlying the network. The set of policies determines the agent's movement on the graph structure. Each policy may be optimised by training to increase the likelihood of navigating through the graph structure to a training target or object entity that is fitted to establish a probability distribution over the set of policies.

Accordingly, DOLPHIN enables the agent to learn paths to the set of targets related to a disease, and attempt to distil this knowledge into the policy network that is generally representative of the set of targets. DOLPHIN also uses more memory-efficient representations to avoid truncating the action space of the agent to highly-connected neighbours. Finally, DOLPHIN considers all possible hops on the agent's last step in order to identify paths to the training targets faster, improving the learning speed over typical reinforcement learning approaches.

A set of targets associated with a query can be generated by a family of policies or a policy network that describes the movement of the agent through a graph structure, along a path starting from the query subject entity (s_{q}). The policy network comprises a latent policy distribution. Specifically, the policy network accepts the input information (s_{q} and its relation r_{q}) of a query and applies its latent policy distribution to select actions during inference to generate the set of objects (o_{q}) that is associated with the query in the training set. Specifically, the training set is received by the policy network as a training triple or example that comprises the subject entity (s_{q}), relation (r_{q}) and training target (o_{q}). An agent traverses the policy network based on the selected actions directed by the policy network with its objective that corresponds to maximizing the log-likelihood of generating the training targets (o_{q}) or the set of target objects across its latent policy distribution.

The actions taken by the agent while traversing the graph structure are hops from a particular node to a different node or the same node. There may be single action or multiplicity of actions taken by the agent in the course traversing the graph structure to generate a training target (o_{q}). Each action is taken at a certain time step or step at a particular point in time. The step may comprise one or more actions that an agent could take or actions available to the agent at the time point. For example, the agent takes exactly one action at each time step. This action may include a hop to another node or a hop back to its current node, based on the probabilities of one or more actions obtained from the policy network. Policy network, therefore, provides probabilities of taking one or more actions at a time step on the graph structure based on the latent policy distribution.

During inference, a search may be performed on the policy network to generate sample targets for a given query subject and relation (s_{q}, r_{q}) pair. Specifically, the search performed in relation to a P network or referred to as the second policy. The search may comprise any one or more heuristic search algorithm that explores a graph structure by expanding the most promising node in a finite set. The heuristic search algorithm may include but are not limited to a best-first search or more specifically, a beam search.

A latent policy distribution underlying the policy network is associated with both the first (Q network) and second policy (P network). The agent uses both networks, P and Q. The Q network considers the query (s_{q}, r_{q}) and the target objects (o_{q}), along with any previous entities and relations in the path that the agent has traversed. Specifically, The Q network is used by the agent to take actions in the network during training. The P network, on the other hand, takes all of the same information except o_{q}. In other words, the P network is unaware of the answer to the query. The P network is not used directly during training to select actions, but it is trained by minimizing its KL divergence from the Q network on each step. The P network is used to select actions during the inference stage. An example formulation of the latent policy distribution associated with the Q and P networks are as follows:

$${\sum }_{q ∈ T} {\sum }_{{o}_{q} ∈ {𝒪}_{q}} ln P ( {o}_{q} \vert {s}_{q} , {r}_{q} ) = {\sum }_{q ∈ T} {\sum }_{{o}_{q} ∈ {𝒪}_{q}} ln \int P ( {o}_{q} \vert {s}_{q} , {r}_{q} , z ) P ( z \vert {s}_{q} , {r}_{q} ) dz$$

where notation T is the set of all queries in the training set, s_{q} and r_{q} represent the subject and relation of a query (e.g. s_{q}=“Ulcerative Colitis” and r_{q}=“HasBenchmarkGeneRelation”), o_{q} is the set of objects associated with the query in the training set (e.g. o_{q}={IL1B, SIRT1 . . . etc.}, the training targets), and z is a latent variable.

In accordance with the example formulation, a distribution attributed to Q network can be used in order to make the optimisation tractable. P and Q networks can be modelled with function approximators parameterised by θ and ψ, respectively. For a single training triple {s_{q}, r_{q}, o_{q}}, the Evidence Lower Bound (ELBO) can be maximised. By doing so, the posterior and prior networks may be dropped out naturally. For example, in the case of variational autoencoders, to learn how to generate the training data is equivalent to learn a prior distribution and learn a posterior distribution. The posterior network, in practice, learns how to generate specific training examples for a given query, and the prior network learns the common patterns across training examples for the given query. Prior network may be used at inference time.

Sampled latent variable z is taken to represent a K-step path [(l₁, e₁), (l₂, e₂) . . . (l_{K}, e_{K})] through the graph structure, where the entity eᵢ is related to the entity eᵢ₋₁ via the link (relation) li. In other words, the latent variables z presents the time step of a path traversing the graph structure from a start node to a target node, where the path may start at the query subject entity (i.e. e₀=s_{q}) and end on e_{K} (e.g. a possible gene target). As shown below, the policy network π_{ψ} to represent Q_{ψ}, where the policy network gives the probability of taking action on each step.

$$z = [ ( {l}_{1} , {e}_{1} ) , ( {l}_{2} , {e}_{2} ) , … ( {l}_{K} , {e}_{K} ) ] \\ {Q}_{ψ} ( z \vert {s}_{q} , {r}_{q} , {o}_{q} ) = {{\prod }^{K}}_{i = 1} {π}_{ψ} ( ( {l}_{i} , {e}_{i} ) \vert {\{ ( {l}_{j} , {e}_{j} ) \}}_{j < i} , {s}_{q} , {r}_{q} , {o}_{q} )$$

The likelihood under the policy can then be expressed as

$${\sum }_{q ∈ T} {\sum }_{{o}_{q} ∈ {𝒪}_{q}} ln P ( {o}_{q} \vert {s}_{q} , {r}_{q} ) = {\sum }_{q ∈ T} {\sum }_{{o}_{q} ∈ {𝒪}_{q}} ln \int P ( {o}_{q} \vert {s}_{q} , {r}_{q} , z ) P ( z \vert {s}_{q} , {r}_{q} ) dz \\ ln P ( {o}_{q} \vert {s}_{q} , {r}_{q} ) \geq {𝔼}_{z ∼ {Q}_{ψ}} [ ln {P}_{θ} ( {o}_{q} \vert {s}_{q} , {r}_{q} , z ) ] - {𝒟}_{KL} [ {Q}_{ψ} ( z \vert {s}_{q} , {r}_{q} , {o}_{q} ) ‖ {P}_{θ} ( z \vert {s}_{q} , {r}_{q} ) ] \\ ln P ( {o}_{q} \vert {s}_{q} , {r}_{q} ) \geq {𝔼}_{z ∼ {Q}_{ψ}} [ ln {δ}_{{e}_{K} {o}_{q}} \vert {s}_{q} , {r}_{q} , z ] - {𝒟}_{KL} [ {Q}_{ψ} ( z \vert {s}_{q} , {r}_{q} , {o}_{q} ) ‖ {P}_{θ} ( z \vert {s}_{q} , {r}_{q} ) ] \\ where \text{?} = \{ \begin{matrix}1 , if {e}_{K} = {o}_{q} \\ 0 , if {e}_{K} \neq {o}_{q}\end{matrix} \\ \text{?} \text{indicates text missing or illegible when filed}$$

In the above formulation, the first expectation on the right side of inequality can be optimised by sampling a path through the graph (based on the latent variables z) from Q, and optimising Q to increase the probability of arriving at the training example o_{q} at the end of the K-step path. This may be achieved by using one or more ML model herein described or by applying, for example, a policy-based reinforcement learning model. In the example, the reinforcement learning model may be parameterised as a two-layer feedforward network with ReLU nonlinearity, which takes in the training example, and outputs a probability distribution over the possible actions from which a discrete action is sampled such that optimal parameters can be found to maximise the expected reward. As an option, smoothing techniques may be applied, i.e. by replacing the at least one training target with one or more different targets sampled uniformly from targets of the graph structure, to maximise the expected reward.

Further to the above formulation, in the case where the term will be ln(0) for paths that do not reach the training example, one way to address this is to apply a form of label smoothing: rather than using an all-or-nothing delta function, we assume that there is a small probability c that the current training target is incorrect (i.e. as a result of noise in the data), and should be replaced by some other target entity selected uniformly from the (Nₑ−1) other potential targets in the graph structure. Effectively, label smoothing replaces training target with one or more different targets sampled uniformly from targets of the graph structure. Hence, the first term in the ELBO becomes:

$${𝔼}_{z ∼ {Q}_{ψ}} [ ( {δ}_{{e}_{K} {o}_{q}} ln ( 1 - ϵ ) + ( 1 - {δ}_{{e}_{K} {o}_{q}} ) ( ln \frac{ϵ}{{N}_{e} - 1} ) ) \vert {s}_{q} , {r}_{q} , z ] = ln \frac{( 1 - ϵ ) ( {N}_{e} - 1 )}{ϵ} {𝔼}_{z ∼ {Q}_{ψ}} [ {δ}_{{e}_{K} {o}_{q}} \vert {s}_{q} , {r}_{q} , z ] + ln \frac{ϵ}{{N}_{e} - 1} .$$

The constant term drops out of the objective function while the multiplier can be absorbed into the learning rate and a new hyperparameter β, giving the new objective function, where

$$J ( θ , ψ ) = {𝔼}_{z ∼ {Q}_{ψ}} [ {δ}_{{e}_{K} {o}_{q}} \vert {s}_{q} , {r}_{q} , z ] - β {𝒟}_{KL} [ {Q}_{ψ} ( z \vert {s}_{q} , {r}_{q} , {o}_{q} ) ‖ {P}_{θ} ( z \vert {s}_{q} , {r}_{q} ) ]$$

The Q network, therefore, corresponds to any encoder or generative model that may include but are not limited to types of deep neural networks (i.e. variational autoencoders for encoding). Applying the Q network generates a z latent variables (and a path through the graph structure) that are conditioned on a full training triplet (including the current training target). The policy network is configured to receive information about which target is currently rewarded. Intuitively, the policy network is given enough information to learn a different policy for each triple in the training set; however, the second loss term applies a bottleneck that pressures all of the policies associated with a query to be similar to a query-specific prior P network. As described below, this prior P can be partially learned and partially fixed, allowing for a form of regularisation.

$${P}_{θ} ( z \vert {s}_{q} , {r}_{q} ) = {{\prod }^{K}}_{i = 1} {π}_{θ} ( ( {l}_{i} , {e}_{i} ) \vert {\{ ( {l}_{j} , {e}_{j} ) \}}_{j < i} , {s}_{q} , {r}_{q} )$$

This prior P network or the second policy may be generated by optimising the first policy. Specifically, the entropic difference between the set of policies in Q network may be calculated or minimised. Techniques such as Kullback-Leibler divergence may be applied to retain this entropic difference minimisation. Subsequently, a beam search may be unrolled at inference time via the prior network P to generate sample targets for a given query subject and relation {s_{q}, r_{q}} while taking the same structure as the Q network, but unconditioned on a specific object.

In the case of an exploration problem, where the agent either rapidly overfits to a single path that reaches a particular query object or fails to sample enough paths to find the query object at all, a regularised prior P or coined herein as policy regularisation improvement may be used as shown:

P_{θ}(z|s_{q},r_{q})=(1−α)P′_{θ}(z|s_{q},r_{q})+αU(z|s_{q},r_{q})

Here, the notation U corresponds to a uniform distribution over all possible actions at each step in the path, and P prime is the policy otherwise generated by the prior network P. By increasing the value of a will induce the agent to follow a more uniform policy and avoid overfitting to a small number of possible actions when applying such entropy-regulated rewards. In addition, through regularisation of the P network, the effective of overfitting and non-convergence shall be ameliorated.

More specifically, applying entropy-regulated rewards (or information-theoretic entropy), for example, is one way to solve the posed exploration/exploitation problem of rapid overfitting of the network to a single path that reaches a particular query object, or the trade-off where not enough paths, to find the query object, have been sampled. The rewards encourage the agent to explore the network, where applied step-wise. That is a multiplicity of actions available to the agent at each step. For instance, flat (high-entropy) policies tend to assign “more” equal probability to all actions; therefore, by rewarding entropy, the agent is rewarded for assigning “more” equal probability to all actions. This prevents the agent from assigning too much probability to particular actions early in training before fully exploring the space (i.e. overfitting).

Moreover, the number of actions available to the agent at a particular step is constant across the state space; i.e. the set of actions that the agent can take is always the same no matter where the agent is situated. On the network or graph, however, the number of actions depends on the number of links connected to the current node. Since entropy scales the number of actions available, this can ascertain that the agent is indirectly rewarded for visiting highly-connected nodes rather than having a flat policy. To avoid this, regularisation of the P network may be applicable.

Furthermore, for more stable and faster learning, the policy gradient loss can be stabilised by (1) subtracting a learned state-dependent baseline estimate of the expected rewards (a common approach in policy gradient methods) or otherwise known herein as policy gradient stabilisation, and (2) evaluating the full expectation of the reward across the action space on the last step of the path. The (2) latter procedure is coined herein as full-expectation rollouts. By considering the outcome of all possible actions on the last step, the agent can find paths that lead to a given target much faster through this full-expectation rollouts.

Tensors may also be allowed to have variable-length dimensions to avoid the truncation of the action space to a pre-defined maximum value. Thus, the memory requirements depend on the average node out-degree across a mini-batch rather than the maximum node out-degree. This could be achieved by borrowing techniques from frameworks such as TensorFlow ragged tensor technique or any other customisable techniques for working with tensors. Any out-of-memory errors that may arise could be addressed by decreasing the mini-batch size and potentially reintroducing action space truncation in very highly-connected networks. In effect, this permits the unconstrained action space or the concept thereof.

ML model(s), predictive algorithms and/or techniques may be used to generate a trained model such as, without limitation, for example one or more trained ML models or classifiers based on input data referred to as training or annotated data associated with ‘known’ entities and/or entity types and/or relationships therebetween derived from large scale datasets (e.g. a corpus or set of text/documents or unstructured data). The input data may also include graph-based statistics as described in more detail in the following sections. With correctly annotated training datasets in such fields as, without limitation, for example chem(o)informatics and bioinformatics, techniques can be used to generate further trained ML models, classifiers, and/or analytical models for use in downstream processes such as, by way of example but not limited to, drug discovery, identification, and optimisation and other related biomedical products, treatment, analysis and/or modelling in the informatics, chem(o)informatics and/or bioinformatics fields. The term ML model is used herein to refer to any type of model, algorithm or classifier that is generated using a training data set and one or more ML techniques/algorithms and the like.

Examples of ML model/technique(s), structure(s) or algorithm(s) for generating a policy network that may be used by the invention as described herein may include or be based on, by way of example only but is not limited to, one or more of: any ML technique or algorithm/method that can be used to generate a trained model based on a labelled and/or unlabelled training datasets; one or more supervised ML techniques; semi-supervised ML techniques; unsupervised ML techniques; linear and/or non-linear ML techniques; ML techniques associated with classification; ML techniques associated with regression and the like and/or combinations thereof. Some examples of ML techniques/model structures may include or be based on, by way of example only but is not limited to, one or more of active learning, multitask learning, transfer learning, neural message parsing, one-shot learning, dimensionality reduction, decision tree learning, association rule learning, similarity learning, data mining algorithms/methods, artificial neural networks (NNs), autoencoder/decoder structures, deep NNs, deep learning, deep learning ANNs, inductive logic programming, support vector machines (SVMs), sparse dictionary learning, clustering, Bayesian networks, types of reinforcement learning, representation learning, similarity and metric learning, sparse dictionary learning, genetic algorithms, rule-based machine learning, learning classifier systems, and/or one or more combinations thereof and the like.

Annotated or labelled training dataset(s) of the above may include but are not limited to, for example, the network of millions of nodes corresponding to diseases, biological processes, pathways and potential therapeutic targets. They are extracted from structured data sources and literature via natural language processing or other data mining techniques. The agent is trained on this dataset(s) to generate multi-hop paths between diseases and targets that predict whether a target has a well-known therapeutic relationship with a disease (or reduced to practice). These multi-hop paths travel through relationships in the graph corresponding to the biological processes and pathways related to the disease, as well as the known protein-protein interactions between the underlying targets. At inference time model generates paths to predict new, reduced to practice, relationships that aren't yet present in the data, which correspond to promising therapeutic targets.

A graph (data) structure comprises a finite set of nodes and a set of edges connecting them. A knowledge graph is a specific embodiment of the graph structure. That is, the knowledge graph and/or entity-entity graph may comprise or represent a graph data structure including a plurality of entity nodes in which each entity node is connected to one or more entity nodes of the plurality of entity nodes by one or more corresponding relationship edges, in which each relationship edge includes data representative of a relationship between a pair of entities. The term knowledge graph, entity-entity graph, entity-entity knowledge graph, graph, or graph dataset may be used interchangeably throughout this disclosure.

An entity may comprise or represent any portion of information or a fact that has a relationship with another portion of information or another fact. The entities of a particular query or query input may include but are not limited to the subject entity (s_{q}), a relation entity (r_{q}), an object entity (o_{q}). These entities may be affiliated with a particular domain or knowledge base. For example, in the biological, chem(o)informatics or bioinformatics space(s) or the corresponding knowledge base thereof, an entity may comprise or represent a biological entity such as, by way of example only but is not limited to, a disease, gene, protein, compound, chemical, drug, biological pathway, biological process, anatomical region or entity, tissue, cell-line, or cell type, or any other biological or biomedical entity and the like. In another example, entities may comprise a set of patents, literature, citations or a set of clinical trials that are related to a disease or a class of diseases. In another example, in the data informatics fields and the like, an entity may comprise or represent an entity associated with, by way of example but not limited to, news, entertainment, sports, games, family members, social networks and/or groups, emails, transport networks, the Internet, Wikipedia pages, documents in a library, published patents, databases of facts and/or information, and/or any other information or portions of information or facts that may be related to other information or portions of information or facts and the like. Entities and relationships may be extracted from a corpus of information such as, by way of example but is not limited to, a corpus of text, literature, documents, web-pages; a plurality of sources (e.g. PubMed, MEDLINE, Wikipedia); distributed sources such as the Internet and/or web-pages, white papers and the like; a database of facts and/or relationships; and/or expert knowledge base systems and the like; or any other system storing or capable of retrieving portions of information or facts (e.g. entities) that may be related to (e.g. relationships) other information or portions of information or facts (e.g. other entities) and the like; and/or any other data source and/or content from which entities, entity types and relationships of interest may be extracted.

For example, in the biological, chem(o)informatics or bioinformatics space(s), graph structure, or more specifically a knowledge graph, may be formed from a plurality of entities in which each entity may represent a biological entity from the group of: from the disease, gene, protein, compound, chemical, drug, biological pathway, biological process, anatomical region or entity, tissue, cell-line, or cell type, clinical trials, any other biological or biomedical entity and the like. Each of the plurality of entities may have a relationship with another one or more entities of the plurality of entities or itself. Thus, a graph structure or a knowledge graph may be formed with entity nodes that include data representative of the entities and relationship edges connecting entities, and further include data representative of the relations/relationships between the entities. The graph structure may include a mixture of different entities with data representative of different relationships therebetween, and/or may include a homogenous set of entities with relationships therebetween.

Although details of the present disclosure may be described, by way of example only but are not limited to, with respect to biological, chem(o)informatics or bioinformatics entities, knowledge graphs and the like are to be appreciated by the skilled person that the details of the present disclosure are applicable as the application demands to any other type of entity, information, data informatics fields and the like. For simplicity, the following describes a specific knowledge graph based on, for example, but is not limited to, gene and disease entities. Another example would be in the domain of online retail, where relationships between customers and products are mapped onto a graph data structure as edges.

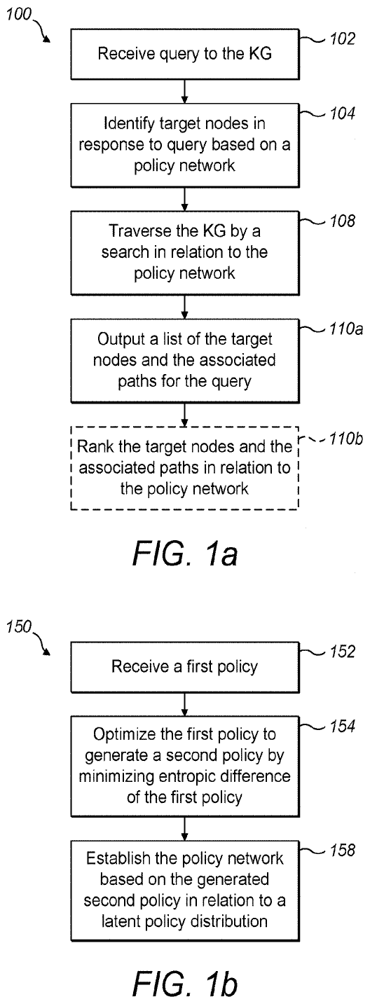

FIG. 1a is a flow diagram illustrating an example process 100 of applying a policy network to determine a list of targets and associated paths according to the invention. A query is first received to the corresponding knowledge graph or a type of structured graph to identify and generate target nodes based on a policy network. A search is conducted to traversing the knowledge graph in relation to the policy network. A list of target nodes and paths associated with the list of target nodes are, in turn, outputted for the query inputted. Optionally, a ranking for the target nodes and associated paths is determined in relation to the policy network. The steps of the process 100 are as follows:

In step 102, the query to the graph structure is received, the query comprises a data representation of at least one query node. The query comprises at least two of a subject entity, a relation entity, an object entity, and a combination of the entities thereof. The subject entity may be a disease entity; the object entity is a target entity of the disease. The relation entity represents the relation between the two. The graph structure may be a knowledge graph that includes a plurality of nodes representing at least a group of entities. In one example, the group of entities may be of at least a group of disease entities and at least a group of target entities. Each of the plurality of nodes are connected via relationship edges to one or more other node(s) of the plurality of nodes, each relationship edge between two node(s) representing a relationship. The knowledge graph may also be associated with a knowledge base pertaining to bioinformatics or chem(o)informatics.

As an option, additional or second input may be received with the query, the second input comprises at least one of a number of time steps, the dimensionality of the vector embedding, or a combination thereof. As another option, the second input may be associated with the query that is defined at training time, before inference, which further comprise at least one hyperparameter. Here, the dimensionality of the vector embedding may be referred to or based on the length of the vectors that stores the actions per time step. The second input is employed when generating the one or more target nodes. At least one hyperparameter may include, for example, but are not limited to the learning rate, batch size, network size, and the like. The number of time steps may correspond to the total number of actions that the agent is allowed to travel within the model or a minimal number of actions could be taken.

In step 104, one or more target nodes in response to the query are identified based on a policy network, where the policy network is configured to determine the one or more target nodes in accordance with a latent policy distribution associated with the policy network. The policy network may be further trained to navigate the knowledge graph from query entities representing disease or biological mechanisms to resultant entities representing targets related to that disease or mechanism. As an option, the policy network may provide probabilities of taking one or more actions at a time step on the graph structure based on the latent policy distribution. Each time step comprises one more action that an agent may take or actions available to the agent at a time point. The agent may take exactly one action at each time step (including, potentially, a hop back to its current node).

Further, the policy network comprises the latent policy distribution. The latent policy distribution may be associated with the P and Q networks together forming the policy network. The latent policy distribution associated with the P network may be used to determine target nodes and associated paths via a heuristic search through the nodes of the graph structure. As an option, penalise may be applied for paths through highly connected node entities in order to include or avoid excluding certain less connected node entities. That is, the associated paths traversing a highly-connected portion of the graph structure may be penalised or even discounted. As such, certain paths through the knowledge graph may be excluded. The graph structure may be a type of knowledge graph that comprises or represents entity data associated with an entity type from the group of: gene; disease; compound/drug; protein; chemical, organ, biological; target; or any other entity type associated with bioinformatics or chem(o)informatics and the like.

In step 108, the graph structure is traversed by a search in relation to the policy network, wherein the search is configured to navigate from the query node to the one or more identified target nodes to determine the associated paths. The search may be, for example, a beam search or any other types of the heuristic searching algorithm that is adapted to search through a graph structure.

In step 110a, a list of the one or more target nodes and the associated paths is outputted for the query. In particular, one or more targets are navigated to via an agent from disease/biological mechanism entities to targets based on the policy network. The paths associated with the one or more target nodes may be identified based on the relation to the query entity (s_{q}) inputted. In the optional step 110b, the list of the one or more target nodes and the associated paths is ranked in relation to the latent policy distribution. Specifically, the ranking may be determined based on or ranked by the estimated probability of navigating along the path according to the learned distribution. As an option, the corresponding associated paths may be selected based on one or more predetermined criteria. The criteria may be determined based on expert knowledge or in relation to excluded detailed paths of step 104. Alternatively and additionally, one or more predetermined criteria may be a restriction or by way of a restrict inference. The restrict inference may be the means to hop across particular subsets of nodes by grouping nodes into families or types. For instance, if the knowledge graph consists nodes corresponding to diseases, biological mechanisms, and targets, restrict inference may be applied to only paths that travel through “biological mechanisms” at inference time to focus on mechanistic reasoning.

Alternatively or additionally, the policy network is regularised (or provided with policy regularisation improvement) in relation to a uniform distribution of all available actions at a time step on the associated paths from the query node to the one or more target nodes. To regularisation to the policy, network mitigates overfitting and promotes the convergence of the network. The regularised network provides a similar probability across all actions taken by the agent. The policy network may further or additionally be governed an action space that comprises every action at a time step stored as one or more variable-length tensors. The one or more variable-length tensors ensures that the agent can learn from all available data under an unconstrained action space. The policy network may additionally or alternatively be stabilised (in terms of policy gradient stabilisation) by accounting for a baseline estimate of an expected reward and an expectation of all available actions (as full-expectation rollouts) at last time step. In such a case, the improved stability also increases the speed for the agent traversing the graph structure.

FIG. 1b is a flow diagram illustrating an example process 150 of training the policy network according to the invention. At the first instance, a first policy is received for generating a policy network from a graph structure. The first policy is optimised to generate a second policy by minimising the entropic difference of the first policy. The policy network is established based on the generated second policy in relation to the underlying latent policy distribution. The steps of process 150 are as follows:

In step 152, the first policy is received for training comprises a set of policies with each policy conditioned on a training triple or example in relation to the graph structure. The set of policies may correspond to a distribution over possible policies fitted using the benchmark training data or query. The training data or query comprises a training triple, which includes the training target. For instance, the training triple may include a subject entity (s_{q}), a relation entity (r_{q}), an object entity (o_{q}). A particular example of the training data or query may include known relationships of disease and gene as a list retrieved from one or more structured databases such as the Comparative Toxicogenomics Database (ctdbase.org) or DisGeNET (disgenet.org), and where the disease and gene may be represented either as a list of (disease, gene) pairs, or alternatively as a set of triples of the form (disease, gene, target).

As an option, the first policy corresponds to a generative model, where the generative model comprises an encoder such as a variational autoencoder. The first policy may be derived or trained via one or more ML model. That is, the probability of arriving at the at least one training target traversing the graph structure is achieved applying the one or more ML models. The one or more ML models may comprise a reinforcement learning model, for example, a policy-based reinforcement learning model and the like.

In step 154, the first policy is optimised to generate a second policy by minimising entropic differences between the set of policies of the first policy. For generate the second policy, techniques such as Kullback-Leibler (KL) divergence may be used. That is, the KL performs argmin of the policies (or distributions) of the posterior distribution (first policy), i.e. find an average distribution, which contributes to the prior distribution (second policy).

Further, the first policy comprises latent variables, where the latent variables present time steps of a path traversing the graph structure from a start node to a target node. The time step of the path may be governed by the first policy. The latent variables may be derived based on the training triple or example embedded in the policy network for generating inferences from the network with a query. By applying the latent variables, the first policy is configured to maximise the probability of arriving at the at least one training target traversing the graph structure starting from a query.

In the case where the probability of arriving at the at least one training target is zero such that no associated paths reach the training example after a finite number of time steps, smoothing may be alternatively or additionally applied by replacing the at last one training target with one or more different targets sampled uniformly from targets of the graph structure.

In step 158, the policy network is established based on the generated second policy in relation to a latent policy distribution. The second policy is derived from the first policy by a minimisation, at each time step, to determine the entropic difference amongst the set of policies in the first policy. That is, the second network is not used directly during training to select actions, but it is trained by minimising its KL divergence from the first network on each time step. In turn, the second policy may be associated with the policy network that is partially fixed to enable a form of regularisation.

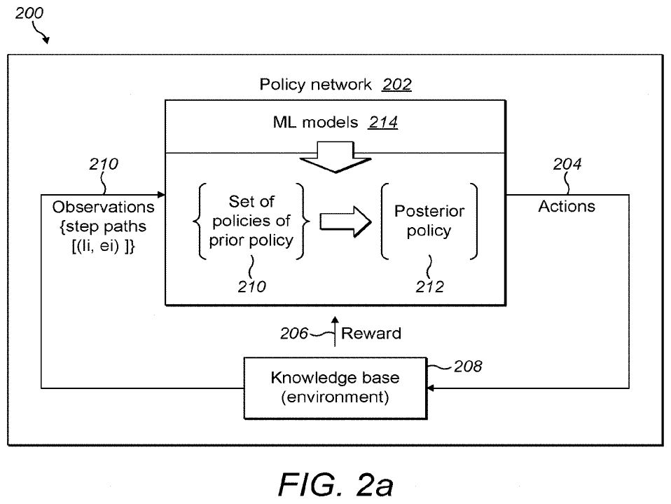

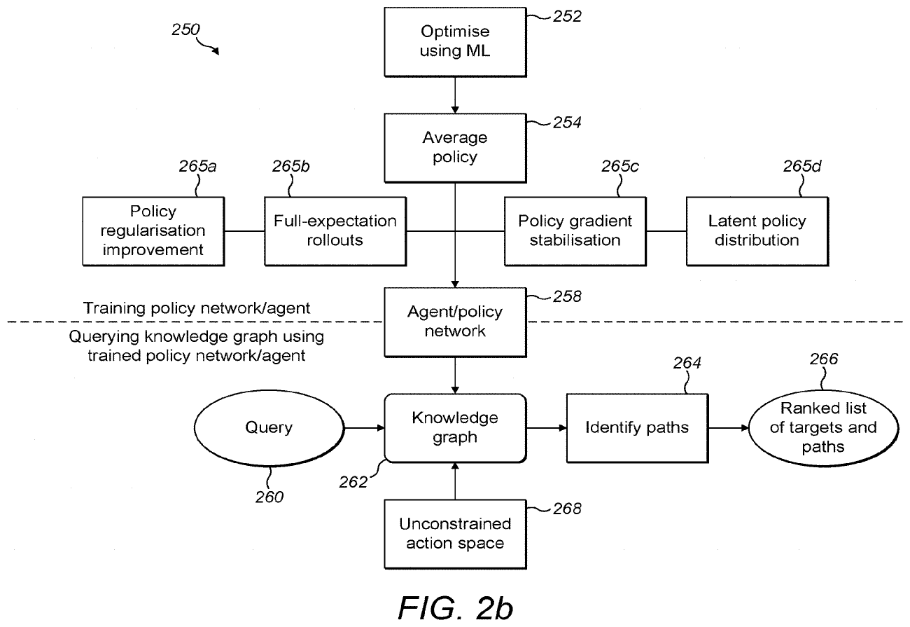

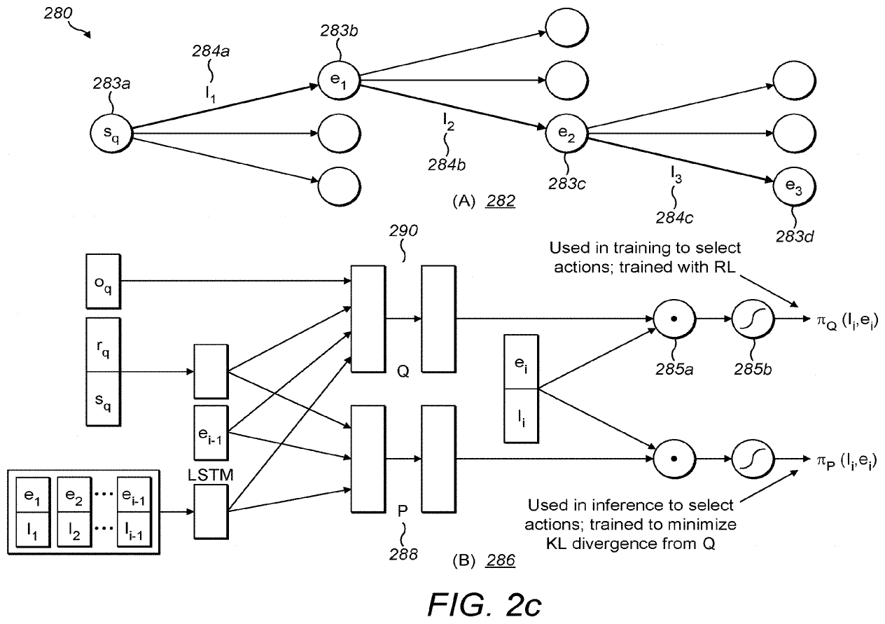

FIG. 2a is a schematic diagram illustrating an example process 200 of the policy network trained and applied to determine a list of targets with associated paths according to the invention. The policy network 202 comprises a set of ML models that is applied directly or indirectly to various policies. These policies include a prior set 210 and a posterior set 212, where the prior set 210 is used to derive the posterior set 212. Based on the policy network 202, a set of actions 204 may be taken by an agent, in the case of reinforcement learning, travel a knowledge base. Specifically, the knowledge base may be in the form of a graph structure or knowledge graph. At each time step, reward (i.e. based on entropy-regulated reward) may be deduced based on a latent probability distribution associated with the policy network that underlies the prior and posterior set of policies. Based on the reward, the agents may act in accordance; in case of a knowledge graph, the agent traverses the graph structure to arrive at specific observations 210 or step paths per action taken. The observations 210 enables the agent to traverse the graph structure while driven by the policy network or more specifically, the latent policy distribution associated with the network. The observations 210 may be applied to train the prior policies 210 in relation to the ML models 214. As an option, regularisation of the prior policies (network) may be applied to further ameliorate the effective of overfitting and non-convergence.

FIG. 2b flow diagram illustrating another example process 250 of the policy network trained via an agent and applied to identify and generate a list of targets with associated paths according to the invention. In this example, policy network 258 or the underlying agent is trained such that based on the trained policy network or agent 258, the inference based on the input query 206 may initiate. Specifically, the input into the knowledge graph 262 is the query relation (e.g. “Disease ______ has therapeutic target ______”). Other inputs include, but are not limited to, hyperparameters (e.g. learning rate), number of hops that the agent can be allowed to travel within the model, and dimensionality of the vector embedding.

In response to the input query (206), an agent (258) or based on the policy network, traverses paths in the knowledge graph (262) according to a policy network (254) to determine its movement. The policy network may be an average policy of the policy network. The average policy may be fitted indirectly by learning a set of policies describing the possible ways to traverse the graph 262. The set of policies can be optimised (103) by training the policy network to increase the likelihood of navigating from known query entities to entities that are known to be related to the query entity via the query relation. This may use reinforcement learning or any other ML algorithm here described that are adapted to receive the training data (s_{q}, r_{q}, and o_{q}).

The agent 258 considers all possible actions such that the agent can find paths that lead to a given target based on the latent policy distribution 265d underlying the policy network 258. In addition, policy gradient stabilisation 256a encourages faster, more stable learning; as well, full-expectation rollouts 256b can be used to ensure that the agent considers all possibilities on the last step to find paths to targets faster. A policy regularisation improvement 265c ensures a similar probability across all actions, and an unconstrained action space 268 ensures that the agent can learn from all available data. Each identified path 264 to a target is added to a list of paths to targets. A ranked list of targets and paths 266 for the input query is outputted.

The procedures or steps for the policy gradient stabilisation 256a, full-expectation rollouts 256b, policy regularisation improvement 265c, and unconstrained action space 268 respectively refers to in this example are further described in detail by the sections.

FIG. 2c is a schematic diagram illustrating another example process 200 of the policy network trained and applied to determine a list of targets with associated paths according to the invention. In the example, part (A) 282 depicts a graph structure with nodes (e.g. s_{q} 283a, e₁ 283b, e₂ 283c, and e₃ 283d) and edges (l₁ 284a, l₂ 284b, and l₃ 284c) forming three layers from the start node labelled as s_{q} 283a. In the context of the policy network, at each step in the path, the agent proceeds along an edge or graph link lᵢ to the next entity eᵢ. The last entity in the path e₃ 283d is the predicted answer to the query (s_{q}, r_{q}). The agent may be rewarded if e₃ 283d is equal to o_{q} or as a suitable answer matching the training data.

Part (b) illustrates an example of a neural network that may be used in relation to training and making an inference based on the policy network. Here, the agent uses two networks, P 288 and Q 290. Yellow layers or unfilled layer are embeddings, green layers or filled layers are neural network RELU layers 285b and the blue circles are operators 285a (a dot for the dot product, a curve for a sigmoid operation).

In particular, the Q network 290 is aware of the query (s_{q}, r_{q}) and the supplied answer to the query o_{q} in the current training example. It is also aware of all previous entities and relations in the path that the agent has visited thus far. The Q network is used by the agent to take actions in the network during training.

The P network 290, on the other hand, takes all of the same information except o_{q}; i.e. it is unaware of the answer to the query in the training example. The P network is not used directly during training to select actions, but it is trained by minimizing its KL divergence from the Q 288 network on each step. The P 290 network is used to select actions during the inference stage.

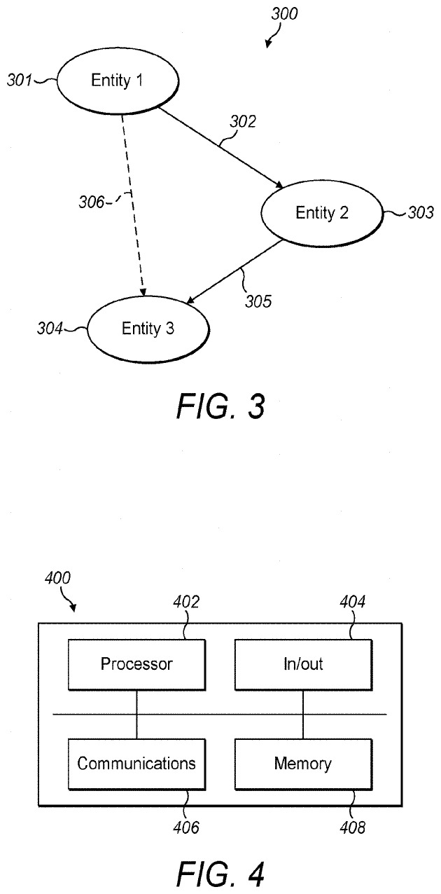

FIG. 3 is a schematic diagram of an example (a portion of) a knowledge graph 300 or subgraph that may be used by the process of figures, la to 2c, according to the invention. The knowledge graph 300 includes a plurality of nodes 301, 303 and (also referred to herein as entity nodes) connected with one or more other nodes to a plurality of edges 302, 305, and 306. The plurality of nodes 301, 303 and 304 represent entities (e.g. Entity 1, Entity 2, Entity 3), which may be, without limitation, for example biological entities and the like, and the plurality of edges 302, 305, and represent relationships that connect the nodes 301, 303 and 304. Each of the edges 302 and 305 may represent a relationship that associates a node of the plurality of nodes 301, 303 and 304 with another of the plurality of nodes 301, 303 and 304. Note, it is also possible to have knowledge graphs in which a node is self-connected by an edge, i.e. an edge that loops back to connect with the same node. Each of the edges 302, 305, and 306 may include further attributes associated with the relationship such as, without limitation, for example directionality, labelling, confidence score of the relationship, and any other useful information associated with the relationship and the like etc. In this example, a first entity node 301 representing a first entity, e.g. Entity 1, is linked via a first edge 302 to a second entity node 303 representing a second entity, e.g. Entity 2, where the first edge 302 is labelled, without limitation, for example with data representing the form of the relationship that exists between the first and second entities, e.g. Entity 1 and Entity 2, of the first and second entity nodes 301 and 303, respectively. For example, in the biomedical domain, the first entity (e.g. Entity 1) of the first entity node 301 may be a gene and the second entity (e.g. Entity 2) of the second entity node 303 may be a disease. Thus, the edge 302 between the first and second entity nodes 301 and 303 may be configured, in this example, to represent a gene—disease relationship, which, without limitation, for example may be tantamount to “causes” if the gene (Entity 1) of the first entity node 301 is responsible for the presence of the disease (Entity 2) of the second entity node 303.

Expanding on this example, the third entity may also be a disease in which shared a disease—disease relationship exists over edge 305 with the second entity. A trained ML model may be configured to examine the knowledge graph and infer new gene-disease relationships to the extent on receiving data representative of a portion or subset of the knowledge graph representing nodes 301, 303 and 304 connected with edges 302 and 305, and based on the connectivity patterns infer or predict a new gene-disease relationship represented by dashed edge 306 between the first entity and the third entity. The new edge 408 may be inferred and incorporated as part of a path forming a subgraph that identifies a potential target node. However, these new inferences may not always prove to be correct, thus, as detailed above, an ML model process the graph-based statistics of various subgraphs to compute scores, when compared to a benchmark dataset or based on one or more criteria determined whether the relationship between the target and the query node may be likely or realistic with high probability.

FIG. 4 is a schematic diagram illustrating an example computing apparatus/system 400 that may be used to implement one or more aspects of the DOLPHIN system(s), apparatus, method(s), and/or process(es) combinations thereof, modifications thereof, and/or as described with reference to FIGS. 1a to 3 and/or as described herein. Computing apparatus/system 400 includes one or more processor unit(s) 402, an input/output unit 404, communications unit/interface 406, a memory unit 408 in which the one or more processor unit(s) 402 are connected to the input/output unit 404, communications unit/interface 406, and the memory unit 408. In some embodiments, the computing apparatus/system 400 may be a server, or one or more servers networked together. In some embodiments, the computing apparatus/system 400 may be a computer or supercomputer/processing facility or hardware/software suitable for processing or performing the one or more aspects of the DOLPHIN system(s), apparatus, method(s), and/or process(es) combinations thereof, modifications thereof, and/or as described with reference to FIGS. 1a to 3 and/or as described herein. The communications interface 406 may connect the computing apparatus/system 400, via a communication network, with one or more services, devices, the server system(s), cloud-based platforms, systems for implementing subject-matter databases and/or knowledge graphs for implementing the invention as described herein. The memory unit 408 may store one or more program instructions, code or components such as, by way of example only but not limited to, an operating system and/or code/component(s) associated with the process(es)/method(s) as described with reference to FIGS. 1a to 3, additional data, applications, application firmware/software and/or further program instructions, code and/or components associated with implementing the functionality and/or one or more function(s) or functionality associated with one or more of the method(s) and/or process(es) of the device, service and/or server(s) hosting the DOLPHIN process(es)/method(s)/system(s), apparatus, mechanisms and/or system(s)/platforms/architectures for implementing the invention as described herein, combinations thereof, modifications thereof, and/or as described with reference to at least one of the FIGS. 1a to 3.

In relation to any of above-described process(es)/method(s) with reference to FIGS. 1a to 3, the DOLPHIN system(s), apparatus, method(s), and/or process(es) suitable for use with the example of computing apparatus described in FIG. 4 is configured to assess relationships amongst the graph nodes by way of utilising an input component, a query component, an extraction component, and an analysis component. The input component is configured to receive the graph, and a query node on the graph and together with a set of connectivity patterns. From the query node, one or more target nodes are identified on the graph by the query component based on the set of connectivity patterns. A subgraph is formed associated with each of the target nodes, where the subgraph comprises multiple paths stemming from the query node to a target node. The extraction component is configured to extract graph-based statistics of a particular subgraph, and the likelihood of predicted relationships between the one or more target nodes and the query node in relation to the subgraph are assessed by the analysis component. The scaffold query tool may apply additionally or alternatively any of the herein described process(es)/method(s) or modules(s)/component(s).

In one aspect is a method for determining one or more target nodes and associated paths from a query of a graph structure, comprising: receiving the query to the graph structure, wherein the query comprises a data representation of at least one query node; identifying one or more target nodes in response to the query based on a policy network, wherein the policy network is configured to determine the one or more target nodes in accordance with a latent policy distribution associated with the policy network; traversing the graph structure by a search in relation to the policy network, wherein the search is configured to navigate from the query node to the one or more identified target nodes to determine the associated paths; and outputting a list of the one or more target nodes and the associated paths for the query, wherein the list are ranked in relation to the latent policy distribution.

In another aspect is a computer-implemented method for generating a policy network from a graph structure for use in the computer implemented method of any preceding claim, the computer-implemented method comprising: receiving a first policy, wherein the first policy comprises a set of policies with each policy conditioned on a training triple in relation to the graph structure; optimising the first policy to generate a second policy by minimising entropic differences between the set of policies of the first policy; and establishing the policy network based on the generated second policy in relation to a latent policy distribution.

In yet another aspect is policy network derived from a knowledge graph conditioned on at least one subject entity and a relation entity to generate one or more object entities from the computer-implemented method according to any one or more of the below options.

In yet another aspect is an apparatus for determining a ranked list of targets and associated paths, the apparatus comprising: an input component configured to receive a query to the graph structure, wherein the query comprises a data representation of at least one query node; a processing component configured to identify one or more target nodes in response to the query based on a policy network, wherein the policy network is configured to determine the one or more target nodes in accordance with a latent policy distribution associated with the policy network; a reactive component configured to traverse the graph structure by a search in relation to the policy network, wherein the search is configured to navigate from the query node to the one or more identified target nodes to determine the associated paths; and an output component configured to output a list of the one or more target nodes and the associated paths for the query, wherein the list are ranked in relation to the latent policy distribution. The apparatus is configured to implement any one or more of the below options.

An apparatus comprising a processor, a memory and a communication interface, the processor connected to the memory and communication interface, wherein the apparatus is adapted or configured to implement the computer-implemented method according to any one or more of the below options.

As an option, the policy network provides probabilities of taking one or more actions at a time step on the graph structure based on the latent policy distribution.

As another option, the policy network is regularised in relation to a uniform distribution of all available actions at a time step on the associated paths from the query node to the one or more target nodes.

As yet another option, the policy network is stabilised by accounting for a baseline estimate of an expected reward and an expectation of all available actions at last time step.

As yet another option, the policy network governs an action space that comprises every action at a time step stored as one or more variable-length tensors.

As another option, the associated paths traversing a highly-connected portion of the graph structure are penalised in relation to the regularised policy network.

As yet in another option further comprises outputting a list of the one or more target nodes and the associated paths for the query further comprises selecting the associated paths based on one or more predetermined criteria.

As yet another option, the search comprises a beam search.

As yet in another option further comprises receiving a second input with the query, wherein the second input is defined at training time

As yet another option, the second input comprises at least one of a number of time steps, dimensionality of the vector embedding, or a combination thereof.

As yet another option, the second input is employed when generating the one or more target nodes.

As yet another option, the first policy corresponds to a generative model.

As yet another option, the generative model comprises an encoder of a variational autoencoder.

As yet another option, the first policy comprises latent variables, wherein the latent variables presents time steps of a path traversing the graph structure from a start node to a target node.

As yet another option, the time steps of a path are governed by the first policy.

As yet another option, the first policy is configured to maximise probability of arriving at the at least one training target traversing the graph structure starting from a query.

As yet another option, the probability of arriving at the at least one training target is zero such that no associated paths reach the training example after finite number of time steps, smoothing is applied by replacing the at last one training target with one or more different targets sampled uniformly from targets of the graph structure.

As yet another option, the probability of arriving at the at least one training target traversing the graph structure is achieved using one or more machine learning models.

As yet another option, the one or more machine learning models comprises a policy-based reinforcement learning model.

The computer-implemented method as claimed in any of claims 10 to 18, wherein minimising entropic differences between the set of policies of the first policy is derived using Kullback-Leibler divergence.

As yet another option, the second policy associated with the policy network is partially fixed to enable a form of regularisation.

As yet another option, the graph structure is a knowledge graph associated with a knowledge base.

As yet another option, the query or the training triple comprises at least two of a subject entity, a relation entity, an object entity, and a combination of the entities thereof.

As yet another option, the query or the training triple is a disease-target based query comprising a disease subject entity, a target object entity, and a relation entity representing a relation therebetween.

As yet another option, policy network comprises a latent policy distribution for an agent to traverse the knowledge graph to determine the one or more object entities by maximising the expected reward received when navigating from an entities to an associated entities based on a received query to the knowledge graph.

As yet another option, the graph structure or knowledge graph comprises a plurality of nodes representing at least a group of entities, wherein each of the plurality of nodes are connected via relationship edges to one or more other node(s) of the plurality of nodes, each relationship edge between two node(s) representing a relationship.

As yet another option, the policy network is trained to navigate the knowledge graph from query entities representing disease or biological mechanisms to result entities representing targets related to that disease or mechanism.

As yet another option, the entities of the graph structure further comprise or represents entity data associated with an entity type from the group of: gene; disease; compound/drug; protein; chemical, organ, biological; target; or any other entity type associated with bioinformatics or chem(o)informatics and the like.

In the embodiments, examples, and aspects of the invention as described above such as process(es), method(s), system(s) and/or tool for querying a graph data structure via the DOLPHIN may be implemented on and/or comprise one or more cloud platforms, one or more server(s) or computing system(s) or device(s). A server may comprise a single server or network of servers; the cloud platform may include a plurality of servers or network of servers. In some examples the functionality of the server and/or cloud platform may be provided by a network of servers distributed across a geographical area, such as a worldwide distributed network of servers, and a user may be connected to an appropriate one of the network of servers based upon a user location and the like.

The above description discusses embodiments of the invention with reference to a single user for clarity. It will be understood that in practice, the system may be shared by a plurality of users, and possibly by a very large number of users simultaneously.

The embodiments described above may be configured to be semi-automatic and/or are configured to be fully automatic. In some examples a user or operator of the querying system(s)/process(es)/method(s) may manually instruct some steps of the process(es)/method(es) to be carried out.

The described embodiments of the invention a system, process(es), method(s) and/or tool for querying a graph data structure and the like according to the invention and/or as herein described may be implemented as any form of a computing and/or electronic device. Such a device may comprise one or more processors which may be microprocessors, controllers or any other suitable type of processors for processing computer executable instructions to control the operation of the device in order to gather and record routing information. In some examples, for example where a system on a chip architecture is used, the processors may include one or more fixed function blocks (also referred to as accelerators) which implement a part of the process/method in hardware (rather than software or firmware). Platform software comprising an operating system or any other suitable platform software may be provided at the computing-based device to enable application software to be executed on the device.

Various functions described herein can be implemented in hardware, software, or any combination thereof. If implemented in software, the functions can be stored on or transmitted over as one or more instructions or code on a computer-readable medium or non-transitory computer-readable medium. Computer-readable media may include, for example, computer-readable storage media. Computer-readable storage media may include volatile or non-volatile, removable or non-removable media implemented in any method or technology for storage of information such as computer-readable instructions, data structures, program modules or other data. A computer-readable storage media can be any available storage media that may be accessed by a computer. By way of example, and not limitation, such computer-readable storage media may comprise RAM, ROM, EEPROM, flash memory or other memory devices, CD-ROM or other optical disc storage, magnetic disc storage or other magnetic storage devices, or any other medium that can be used to carry or store desired program code in the form of instructions or data structures and that can be accessed by a computer. Disc and disk, as used herein, include compact disc (CD), laser disc, optical disc, digital versatile disc (DVD), floppy disk, and blu-ray disc (BD). Further, a propagated signal is not included within the scope of computer-readable storage media. Computer-readable media also includes communication media including any medium that facilitates transfer of a computer program from one place to another. A connection or coupling, for instance, can be a communication medium. For example, if the software is transmitted from a website, server, or other remote source using a coaxial cable, fiber optic cable, twisted pair, DSL, or wireless technologies such as infrared, radio, and microwave are included in the definition of communication medium. Combinations of the above should also be included within the scope of computer-readable media.

Alternatively, or in addition, the functionality described herein can be performed, at least in part, by one or more hardware logic components. For example, and without limitation, hardware logic components that can be used may include Field-programmable Gate Arrays (FPGAs), Program-specific Integrated Circuits (ASICs), Program-specific Standard Products (ASSPs), System-on-a-chip systems (SOCs). Complex Programmable Logic Devices (CPLDs), etc.

Although illustrated as a single system, it is to be understood that the computing device may be a distributed system. Thus, for instance, several devices may be in communication by way of a network connection and may collectively perform tasks described as being performed by the computing device.

Although illustrated as a local device it will be appreciated that the computing device may be located remotely and accessed via a network or other communication link (for example using a communication interface).

The term ‘computer’ is used herein to refer to any device with processing capability such that it can execute instructions. Those skilled in the art will realise that such processing capabilities are incorporated into many different devices and therefore the term ‘computer’ includes PCs, servers, IoT devices, mobile telephones, personal digital assistants and many other devices.

Those skilled in the art will realise that storage devices utilised to store program instructions can be distributed across a network. For example, a remote computer may store an example of the process described as software. A local or terminal computer may access the remote computer and download a part or all of the software to run the program. Alternatively, the local computer may download pieces of the software as needed, or execute some software instructions at the local terminal and some at the remote computer (or computer network). Those skilled in the art will also realise that by utilising conventional techniques known to those skilled in the art that all, or a portion of the software instructions may be carried out by a dedicated circuit, such as a DSP, programmable logic array, or the like.

It will be understood that the benefits and advantages described above may relate to one embodiment or may relate to several embodiments. The embodiments are not limited to those that solve any or all of the stated problems or those that have any or all of the stated benefits and advantages. Variants should be considered to be included into the scope of the invention.

Any reference to ‘an’ item refers to one or more of those items. The term ‘comprising’ is used herein to mean including the method steps or elements identified, but that such steps or elements do not comprise an exclusive list and a method or apparatus may contain additional steps or elements.

As used herein, the terms “component” and “system” are intended to encompass computer-readable data storage that is configured with computer-executable instructions that cause certain functionality to be performed when executed by a processor. The computer-executable instructions may include a routine, a function, or the like. It is also to be understood that a component or system may be localized on a single device or distributed across several devices. Further, as used herein, the term “exemplary”, “example” or “embodiment” is intended to mean “serving as an illustration or example of something”. Further, to the extent that the term “includes” is used in either the detailed description or the claims, such term is intended to be inclusive in a manner similar to the term “comprising” as “comprising” is interpreted when employed as a transitional word in a claim.

The figures illustrate exemplary methods. While the methods are shown and described as being a series of acts that are performed in a particular sequence, it is to be understood and appreciated that the methods are not limited by the order of the sequence. For example, some acts can occur in a different order than what is described herein. In addition, an act can occur concurrently with another act. Further, in some instances, not all acts may be required to implement a method described herein.

Moreover, the acts described herein may comprise computer-executable instructions that can be implemented by one or more processors and/or stored on a computer-readable medium or media. The computer-executable instructions can include routines, sub-routines, programs, threads of execution, and/or the like. Still further, results of acts of the methods can be stored in a computer-readable medium, displayed on a display device, and/or the like.

The order of the steps of the methods described herein is exemplary, but the steps may be carried out in any suitable order, or simultaneously where appropriate. Additionally, steps may be added or substituted in, or individual steps may be deleted from any of the methods without departing from the scope of the subject matter described herein. Aspects of any of the examples described above may be combined with aspects of any of the other examples described to form further examples without losing the effect sought.

It will be understood that the above description of a preferred embodiment is given by way of example only and that various modifications may be made by those skilled in the art.

What has been described above includes examples of one or more embodiments. It is, of course, not possible to describe every conceivable modification and alteration of the above devices or methods for purposes of describing the aforementioned aspects, but one of ordinary skill in the art can recognize that many further modifications and permutations of various aspects are possible. Accordingly, the described aspects are intended to embrace all such alterations, modifications, and variations that fall within the scope of the appended claims.

1. A computer-implemented method for determining one or more target nodes and associated paths from a query of a graph structure, comprising:
receiving the query to the graph structure, wherein the query comprises a data representation of a query node;
identifying one or more target nodes in response to the query based on a policy network, wherein the policy network is configured to determine the one or more target nodes in accordance with a latent policy distribution associated with the policy network;
traversing the graph structure by a search in relation to the policy network, wherein the search is configured to navigate from the query node to the one or more target nodes to determine the associated paths; and
outputting a list of the one or more target nodes and the associated paths for the query, wherein the list are ranked in relation to the latent policy distribution.

2. The computer-implemented method of claim 1, wherein the policy network provides probabilities of taking one or more actions at a time step on the graph structure based on the latent policy distribution.

3. The computer-implemented method as claimed in claim 1, wherein the policy network is regularised in relation to a uniform distribution of all available actions at a time step on the associated paths from the query node to the one or more target nodes.

4. The computer-implemented method as claimed in claim 1, wherein the policy network is stabilised by accounting for a baseline estimate of an expected reward and an expectation of all available actions at last time step.

5. The computer-implemented method as claimed in claim 1, wherein the policy network governs an action space that comprises every action at a time step stored as one or more variable-length tensors.

6. The computer-implemented method as claimed in claim 1, wherein the associated paths traversing a highly connected portion of the graph structure are penalised in relation to the policy network.

7. The computer-implemented method as claimed in claim 1, wherein outputting a list of the one or more target nodes and the associated paths for the query further comprises selecting the associated paths based on one or more predetermined criteria.

8. The computer-implemented method as claimed in claim 1, wherein the search comprises a beam search.

9. The computer-implemented method as claimed in claim 1, further comprising: receiving a second input with the query, wherein the second input is defined at training time; wherein the second input comprises at least one of a number of time steps, dimensionality of a vector embedding, or a combination thereof; and wherein the second input is employed when generating the one or more target nodes.

10. A computer-implemented method for generating a policy network from a graph structure for use in the computer-implemented method of claim 1, the computer-implemented method comprising:
receiving a first policy, wherein the first policy comprises a set of policies with each policy conditioned on a training triple in relation to the graph structure;
optimising the first policy to generate a second policy by minimising entropic differences between the set of policies of the first policy; and
establishing the policy network based on the second policy in relation to a latent policy distribution.

11. The computer-implemented method as claimed in claim 10, wherein the first policy corresponds to a generative model; wherein the generative model comprises an encoder of a variational autoencoder.

12. The computer-implemented method as claimed in claim 10, wherein the first policy comprises latent variables, wherein the latent variables presents time steps of a path traversing the graph structure from a start node to a target node; wherein the time steps of a path are governed by the first policy.

13. The computer-implemented method as claimed in claim 10, wherein the first policy is configured to maximise a probability of arriving at at least one training target traversing the graph structure starting from a query; wherein the probability of arriving at the at least one training target traversing the graph structure is achieved using one or more machine learning models; where the one or more machine learning models comprises a policy-based reinforcement learning model.

14. The computer-implemented method as claimed in claim 13, wherein when the probability of arriving at the at least one training target is zero, such that no associated paths reach the training example after finite number of time steps, smoothing is applied by replacing the at least one training target with one or more different targets sampled uniformly from targets of the graph structure.

15. The computer-implemented method as claimed in claim 10, wherein minimising entropic differences between the set of policies of the first policy is derived using Kullback-Leibler divergence.

16. The computer-implemented method as claimed in claim 10, wherein the second policy associated with the policy network is partially fixed to enable a form of regularisation.

17. The computer-implemented method as claimed in claim 10, wherein the graph structure is a knowledge graph associated with a knowledge base; wherein the knowledge graph comprises a plurality of nodes representing at least a group of entities, wherein each of the plurality of nodes are connected via relationship edges to one or more other node(s) of the plurality of nodes, each relationship edge between two node(s) representing a relationship; and wherein entities of the graph structure further comprise or represents entity data associated with an entity type from the group consisting of: gene; disease; compound/drug; protein; chemical, organ, biological; target; or any other entity type associated with bioinformatics or chem(o)informatics.

18. The computer-implemented method according to claim 17, wherein the policy network is trained to navigate the knowledge graph from query entities representing disease or biological mechanisms to result entities representing targets related to that disease or mechanism.

19. The computer-implemented method as claimed in claim 1, wherein the query is a disease-target based query comprising a disease subject entity, a target object entity, and a relation entity representing a relation therebetween.

20. A computer-readable medium comprising computer readable code or instructions stored thereon, which when executed on a processor, causes the processor to implement the computer-implemented method according to claim 1.
